(19) United States
(12) Patent Application Publication
(10) Pub. No.: US 2007/0202135 A1
(43) Pub. Date: Aug. 30, 2007

(54) Ionized compositions and methods of use therefore

(75) Inventor: Richard Ouellette, Poway, CA (US)

Correspondence Address: BUCHANAN, INGERSOLL & ROONEY LLP, P.O. BOX 1404, ALEXANDRIA, VA, 22313-1404, US

(73) Assignee: AnglesBeautyCare Group, Inc., Poway, CA (US)

(21) Appl. No.: 11/361,524

(22) Filed: Feb. 24, 2006

(51) Int. Cl. A61K 8/26

(52) U.S. Cl. 424/401; 424/61; 424/64; 424/70.2

(57) ABSTRACT

This invention relates to methods for preparing an ionic composition suitable for use as a cosmetic product, products containing such compositions, and containers for containing and dispensing such products.

### TECHNICAL FIELD

This invention relates to cosmetic products containing compositions ionically-modified by a complex silicate.

### BACKGROUND

Solid particles, such as complex silicates, have been used as agents in a variety of formulations including cosmetic products. Some complex silicates possess unusual properties. For example, tourmaline is a complex borosilicate mineral that possesses pyroelectric and piezoelectric properties. Tourmaline's unique electrical activity results from the presence of permanent electrodes within the crystalline structure. In particular, tourmaline in water produces an electrical discharge which dissociates the water molecule into hydrogen and hydroxyl ions, which in turn produce hydronium ions (H₃O⁺) and hydrated hydroxyl ion (H₃O₂⁻) by their reactions with H₂O molecules. Both these ions are reported to have surface active properties.

When applied directly to a target surface, solid particles containing complex silicates can impart benefits to the surface. Such benefits include shine, gloss, surface smoothening, and manageability. Typically the particles are applied via preparations that are rubbed, sprayed, or otherwise applied directly onto the surface to be affected. Examples of such preparations include moisturizers, lotions, creams, shampoos and hairsprays.

Despite the benefits that can be delivered through direct application and retention of complex silicates to a surface, it can be desirable to transfer the effect of such particles to a surface without direct contact between the particles and the surface. Accordingly, compositions that have been beneficially modified through contact with a complex silicate and intended for deposition on a target surface are needed.

### SUMMARY

Provided herein are methods for preparing cosmetic products containing compositions ionized through contact with a complex silicate. Accordingly, in one embodiment, a method of preparing an ionic composition suitable for use as a cosmetic product is provided. The method includes contacting a composition including an ionizable solvent with a material that includes a piezoelectric and/or pyroelectric complex silicate. The ionizable solvent is at least partially ionized by the contact. The method further includes separating the composition including the ionized solvent from the material that includes the complex silicate resulting in a composition that is at least partially ionized. The method optionally includes heating the composition containing the complex silicate, under pressure, prior to separation.

In another embodiment, the complex silicate is selected from the tourmaline mineral group (TMG) including those members that have the following general chemical formula:
Na(Al,Fe,Li,Mg,Mn)M₃Al(Si₆O₁₈)(BO₃)₃(OH,F)₄.

In some embodiments, a complex silicate suitable for use in the present compositions include those having a chemical formula selected from:
Na(Li_{1.5},Al_{1.5})Al₆Si₆O₁₈(BO₃)₃(OH)₄;
NaFe²⁺₃Al₆Si₆O₁₈(BO₃)₃(OH)₄;
NaMg₃Al₆Si₆O₁₈(BO₃)₃(OH)₄;
NaMg₃Cr₆Si₆O₁₈(BO₃)₃(OH)₄;
NaAl₃Al₆Si₆O₁₈(BO₃)₃O₃OH;
NaFe³⁺₃Al₆Si₆O₁₈(BO₃)₃O₃F
NaFe³⁺₃(Fe³⁺₄Mg₂Si₆O₁₈(BO₃)₃(OH)₃O;
NaMg₃V₆Si₆O₁₈(BO₃)₃(OH)₄;
Ca(Li₂Al)Al₆Si₆O₁₈(BO₃)₃(OH)₃F;
CaMg₃(MgAl₅Si₆O₁₈(BO₃)₃(OH)₃F;
CaFe²⁺₃(MgAl₅Si₆O₁₈(BO₃)₃(OH)₄;
(LiAl₂)Al₆Si₆O₁₈(BO₃)₃(OH)₄;
(Fe²⁺₂Al)Al₆Si₆O₁₈(BO₃)₃(OH)₄; and
(Mg₂Al)Al₆Si₆O₁₈(BO₃)₃(OH)₄.

In some aspects, complex silicates suitable for use in the present compositions include elbaite, schorl, dravite, chromdravite, olenite, buergerite, povondraite, vanadiumdravite, liddicoatite, uvite, hydroxyferuvite, rossmanite, foitite, and magnesiofoitite.

In one embodiment, the material that includes the complex silicate is a particle. Such particles may be spheroids, cuboids, pyramidal or any other three dimensional shape suitable for a particle associated with a complex silicate as described herein. In some aspects, the particle size ranges from about 0.1 mm to about 100 mm in diameter. In other aspects, the particle size ranges from about 0.1 mm to about 10 mm in diameter. In other aspects, the particle size ranges from about 0.1 mm to about 1 mm in diameter.

In one embodiment, the complex silicate is present in the material in an amount ranging from about 0.50% to about 99.5% by weight, or from about 0.50% to about 50% by weight, or from about 0.50% to about 25.5% by weight.

In other embodiments, a cosmetic product that includes a composition set forth herein includes a hair care product, a skin-care cream, a lotion, a body powder, a body spray, perfume, lipstick, fingernail polish, eye and facial makeup, hair color product, deodorant, bath oil and bubble bath. In some aspects, the hair care product is a shampoo, conditioner, hairspray, permanent wave product or mousse.

In some embodiments, an ionizable solvent includes deionized water or glycol, or derivatives thereof. In one aspect, the ionizable solvent is deionized water.

In other embodiments the composition further includes an amphoteric surfactant, a mild anionic, an anionic sulfate, a humectant, an emollient, a viscosity modifier, a biologically active material, a foam booster, a conditioning agent, a silicone surfactant, a cationic polyquaternium surfactant, an anti-dandruff agent, a fragrance compound, or any combination thereof. Exemplary amphoteric surfactants include alkyl betaines, amido alkyl betaines, sulfobetaines, and N-alkyl-amino propionates. Exemplary anionics include fatty acid carboxylates, taurates, sulfosuccinates, isethionates, taurates, sarcosinates, and monoalkyl phosphates. Exemplary viscosity modifiers include gum, cellulose derivatives, PEG esters or carbopol resins. Exemplary biologically active materials include hydroxy acids, antiinflammatories, and an anti-irritants. Exemplary anionic sulfates include alkyl sulfates, alkyl ether sulfates, monosulfosuccinates, disulfosuccinates, alpha-olefin sulfates, and monoglyceride sulfates. Exemplary foam boosters include alkanolamides and N-alkylpyrrolidones. Exemplary conditioning agents include amine oxides. Exemplary silicone surfactants include amodimethicone and dimethicone copolyol. Exemplary anti-dandruff agents include zinc pyrithione and pyroctone olamlne.

In other embodiments, methods provided herein may further include heating the composition for a period of time ranging from about 10 seconds to about 10 minutes, or for a period of time ranging from about 30 seconds to about 1 minute.

In other embodiments, the composition may be heated at a temperature ranging from about 25° C. to about 75° C., or from about 30° C. to about 60° C., or from about 37° C. to about 50° C.

In yet another embodiment, the composition may be heated under a pressure ranging from about 1 atm to about 5 atm, or from about 1 atm to about 2 atm.

In one embodiment, a cosmetic product that includes an ionic composition prepared by a method described herein, is provided.

In another embodiment, a method for cleansing, conditioning or modifying the appearance of hair, is provided. The method includes contacting the hair with a cosmetic product provided herein under conditions suitable for cleansing, conditioning or modifying the appearance of the hair.

In yet another embodiment, a method of applying a hair care product that includes an ionic composition to the hair, is provided. The method includes preparing an ionic composition provided herein, adding the composition to a hair care product, thoroughly mixing the composition into the hair care product, and applying the hair care product to the hair.

In yet another embodiment, a container for containing and dispensing a cosmetic product is provided. The container includes at least one chamber comprising a composition comprising an ionizable solvent associated with a material comprising a piezoelectric and/or pyroelectric complex silicate, wherein the ionizable solvent is at least partially ionized. The container further includes a mechanism for separating the composition comprising the ionized solvent from the material comprising the complex silicate, wherein the composition is at least partially ionized, and an outlet for dispensing the ionized composition. The mechanism may be as simple as allowing the material to float to the bottom the container before the composition exits the container. Additional mechanisms include manufacturing the exit point (i.e., the outlet) so that it allows the ionized composition to exit the container but not the particles. For example, the outlet may be manufacture in such a way that it is too small to allow the particles to exit the container. Alternatively, the outlet may be operably associated with a conduit that extends in to a chamber. The conduit may be operably associated with a material holder that confines the material but allows the material to contact the composition before exiting.

The details of one or more embodiments of the disclosure are set forth in the accompanying description and examples below. Other features, objects, and advantages will be apparent from the description and examples, and from the claims.

### DETAILED DESCRIPTION

As will be described in more detail below, methods for preparing cosmetic products containing compositions ionized through contact with a complex silicate are provided. Products containing such compositions are also provided. Containers for containing and dispensing the cosmetic product are also provided.

Based, in part, upon information derived from contacting a target surface, such as skin or hair, with a cosmetic product incorporating an ionized composition, the present invention provides methods and compositions for improving the appearance of a target surface. The cosmetic products provided herein utilize the unique electrical properties of complex silicates to achieve these results.

Complex silicates, in combination with other materials, have been suggested for use in connection with the hair and skin treatments. See, e.g., U.S. Pat. No. 6,357,075, U.S. Pat. No. 6,205,674, U.S. Pat. No. 6,036,965, U.S. Pat. No. 5,941,253, U.S. Patent Application Publication No. 2005/0045742, and U.S. Patent Application Publication No. 2005/0208004, the contents of which are incorporated herein by reference in their entirety.

In the above cited publications, the complex silicates are always used in a manner that requires direct contact with the hair or skin. Moreover, in each of these patents, the complex silicates are brought in contact with the hair or scalp during only certain phases or aspects of hair care, i.e., brushing, drying or curling. Thus, contact can only occur during certain very specific activities that may or may not be used by any particular person at any particular time. The methods and compositions provided herein are not so limited.

Provided herein are methods and compositions for transferring the beneficial effects of particles comprised of a complex silicate to a surface without direct contact between the particles and the surface material. An ionizable solvent is modified (e.g., ionized) through contact with a material and the beneficial effects of the contact transferred to the target surface without transferring the complex silicate associated with the material to the target surface. Accordingly, in one embodiment, a method of preparing an ionic composition suitable for use as a cosmetic product is provided. The method includes contacting a composition including an ionizable solvent with a material that includes a complex silicate possessing piezoelectric and/or pyroelectric properties. The ionizable solvent is at least partially ionized by the contact. The method further includes separating the composition including the ionized solvent from the material that includes the complex silicate resulting in a composition that is at least partially ionized. The method optionally includes heating the composition containing the complex silicate, under pressure, prior to separation.

One method for the preparation of the ionized solvent comprises suspending material that includes a complex silicate in a compatible (i.e., ionizable) solvent for a suitable length of time. Optionally, the solvent-silicate combination may be heated and/or subjected to increased pressure. The resulting ionized solvent may be included in a composition that is suitable for application applied to the hair and scalp (e.g., a hair care product). According to one embodiment, the ionized solvent is associated with a hair care product. The hair care product is applied to the hair and scalp such that the ionized solvent contacts the hair and scalp.

A composition comprising an ionizable solvent in contact with a complex silicate includes cosmetic products, such as hair care products or cosmetic cleaning products that would benefit from the unique electrical properties associated with a complex silicate such as tourmaline. The term “cosmetic product, as used herein, includes articles intended to be applied to the human body for cleansing, beautifying, promoting attractiveness, or altering the appearance without affecting the body's structure or functions. This definition includes skin-care creams, lotions, powders and sprays, perfumes, lipsticks, fingernail polishes, eye and facial makeup, permanent waves, hair colors, deodorants, baby products, bath oils, bubble baths, and mouthwashes, as well as any material intended for use as a component of a cosmetic product.

For example, an ionic composition suitable for use as a cosmetic product includes facial cleansing compositions, or hair or body shampoos or cleansers. Depending on the nature of the product, the ionic composition, in addition to including an ionizable solvent comprising a complex silicate, may include ordinarily be combined with other components which aid in cleansing and/or conditioning of the surface to which the composition is applied. In a facial cleanser, for example, additional components can include one or more of the milder surfactants. Examples include, but are not limited to the amphoteric surfactants, such as alkyl betaines, amido alkyl betaines, sulfobetaines, and N-alkyl-amino propionates; or mild anionics, such as fatty acid carboxylates, taurates, sulfosuccinates, isethionates, taurates, and sarcosinates, or monoalkyl phosphates. Other desirable components of facial cleansers can include skin conditioning agents, such as humectants or emollients; viscosity modifiers, such as gums, cellulose derivatives, PEG esters and carbopol resins; and biologically active materials, such as hydroxy acids, antiinflammatories, or anti-irritants. In the latter case, the surface active properties of the tourmaline may enhance delivery of the active agents.

The cosmetic product can also be a hair or body shampoo or cleanser. Like the facial cleanser, hair or body shampoos will contain surfactants or detergents for cleansing. Examples include anionic sulfates, such as alkyl sulfates and alkyl ether sulfates, mono- and disulfosuccinates, alpha-olefin sulfates, and monoglyceride sulfates, as well as those surfactants useful for facial cleansing. Shampoos also will usually contain foam boosters, which are usually nonionic surfactants, such as alkanolamides or N-alkylpyrrolidones; and conditioning agents, such as amine oxides, silicone surfactants such as amodimethicone or dimethicone copolyol, and cationic polyquaternium surfactants. Also frequently added to hair shampoos are active components such as anti-dandruff agents, e.g., zinc pyrithione or pyroctone olamlne. The process for formulation of cleansing products is well known in the art, and additional possible components for such formulations can be found, for example, in The International Cosmetic Ingredient Dictionary and Handbook, Tenth Edition (2004), the contents of which are incorporated herein by reference.

In one example, the cosmetic products benefit from the contacting of an ionizable solvent with a complex silicate because the complex silicate, while not in direct contact with the skin or hair, modifies the solvent such that it may permit a reduction in the amount of detergent-type surfactant necessary to achieve proper cleansing. In addition, the modification may also effective in the process of oil removal from substrates, and therefore, may be particularly suitable for use in cleansers intended for use on oily skin or hair.

As previously noted, methods provided herein include contacting a composition including an ionizable solvent with a material that includes a complex silicate possessing piezoelectric and/or pyroelectric properties. Piezoelectricity and pyroelectricity are properties possessed by certain classes of minerals or synthetic crystalline materials that lack a center of symmetry. Piezoelectricity is the ability of a mineral or crystal to acquire opposing electrical charges on opposing surfaces when mechanical stress (such as bending, stretching, or compression) is applied to the crystal. The piezoelectric effect is caused by the displacement of ionic charges within a crystal structure, and the magnitude of the charge generally is proportional to the amount of stress applied.

Pyroelectricity is the ability of a mineral or crystal to acquire opposing electrical charges on opposing surfaces as a result of heating. Tourmaline was recognized as a distinct mineral with a highly variable composition because of pyroelectricity. Pyroelectricity can be visualized as one side of a triangle, where each corner represents energy states in the crystal: kinetic, electrical and thermal energy. The side between electrical and thermal corners represents the pyroelectric effect and produces no kinetic energy. The side between kinetic and electrical corners represents the piezoelectric effect and produces no heat. Very small changes in temperature can produce an electric potential due to a materials' pyroelectricity. Motion detection devices are often designed around pyroelectric materials, as the heat of a human or animal from several feet away is enough to generate a difference in charge.

Accordingly, polarized substances that charge ambient materials such as ionizable solvents may be used as a complex silicate. Tourmaline is an example of a substance that is polarized. An example of a material comprising a complex silicate includes ceramics containing tourmaline ore.

The tourmaline mineral group is chemically one of the most complicated groups of silicate minerals. It is a silicate of aluminum and boron, but because of isomorphous replacement (solid solution), its composition varies widely with sodium, calcium, iron, magnesium, lithium and other elements entering into the structure. Tourmaline has a variety of polar characteristics reflecting its non-centrosymmetric nature. The non-centrosymmetry results in piezoelectric and pyroelectric responses to changing stresses and temperature, with the intensity of the pyroelectric response being composition-dependent.

Accordingly, tourmaline subgroups, such as dravite, schorl, elbaite, rubellite, indicolite, Brazilian sapphire, verdelite or Brazilian emerald, achroite, chromdravite, olenite, buergerite, povondraite, vanadiumdravite, liddicoatite, uvite, hydroxyferuvite, rossmanite, foitite, and magnesiofoitite, are encompassed by the term “complex silicate.”

A general chemical formula for tourmaline and tourmaline subgroups includes:
General: Na(Al,Fe,Li,Mg,Mn)M₃Al (Si₆O₁₈)(BO₃)₃(OH,F)₄

More specific formulae include:

| | |
|---|---|
| Elbaite: | Na(Li_{1.5},Al_{1.5})Al₆Si₆O₁₈(BO₃)₃(OH)₄ |
| Schorl: | NaFe²⁺₃Al₆Si₆O₁₈(BO₃)₃(OH)₄ |
| Dravite: | NaMg₃Al₆Si₆O₁₈(BO₃)₃(OH)₄ |
| Chromdravite: | NaMg₃Cr₆Si₆O₁₈(BO₃)₃(OH)₄ |
| Olenite: | NaAl₃Al₆Si₆O₁₈(BO₃)₃O₃OH |
| Buergerite: | NaFe³⁺₃Al₆Si₆O₁₈(BO₃)₃O₃F |
| Povondraite: | NaFe³⁺₃(Fe³⁺₄Mg₂Si₆O₁₈(BO₃)₃(OH)₃O |
| Vanadiumdravite: | NaMg₃V₆Si₆O₁₈(BO₃)₃(OH)₄ |
| Liddicoatite: | Ca(Li₂Al)Al₆Si₆O₁₈(BO₃)₃(OH)₃F |
| Uvite: | CaMg₃(MgAl₅Si₆O₁₈(BO₃)₃(OH)₃F |
| Hydroxyferuvite: | CaFe²⁺₃(MgAl₅Si₆O₁₈(BO₃)₃(OH)₄ |
| Rossmanite: | (LiAl₂)Al₆Si₆O₁₈(BO₃)₃(OH)₄ |
| Foitite: | (Fe²⁺₂Al)Al₆Si₆O₁₈(BO₃)₃(OH)₄ |
| Magnesiofoitite: | (Mg₂Al)Al₆Si₆O₁₈(BO₃)₃(OH)₄ |

A material comprising a complex silicate can generally be described as a particle. Such particles may be spheroid, cuboid, pyramidal or any other three dimensional shape suitable for a particle complexed with, or associated with, a complex silicate as described herein. In some aspects, the particle size ranges from about 0.1 mm to about 100 mm in diameter. In other aspects, the particle size ranges from about 0.1 mm to about 10 mm in diameter. In other aspects, the particle size ranges from about 0.1 mm to about 1 mm in diameter.

In one embodiment, the complex silicate is present in the material in an amount ranging from about 0.50% to about 99.5% by weight, or from about 0.50% to about 50% by weight, or from about 0.50% to about 25.5% by weight.

A composition of the invention, such as a hair care product, comprises an ionized solvent. An “ionizable solvent” is a solvent that is relatively non-toxic and is ionizable when contacted by a complex silicate. The solvent may be selected from the group consisting of deionized water, glycol and its derivatives, organic acids and bases and their derivatives, inorganic acids and bases and their derivatives, organic amines, and salt solutions. A water molecule contains the potential forms of all crystals in its primary form of a tetrahedron. Water can bring different forms of ions into a crystalline state an hold them in solution. The more structured the water is, the higher concentration of ions it can hold.

For example, tourmaline's unique electrical activity results from the presence of permanent electrodes within the crystalline structure. Tourmaline in, for example, water produces an electrical discharge which dissociates the water molecule into hydrogen and hydroxyl ions, which in turn produce hydronium ions (H₃O⁺) and hydrated hydroxyl ion (H₃O₂⁻) by their reactions with H₂O molecules. Both these ions are reported to have surface active properties. For example, U.S. Pat. No. 5,599,455 discloses a system for water treatment in which tourmaline is used in an ion generator to generate hydronium and hydroxyl ions in the water, with the reported result of “high surface activity” in the water, which is then recommended for use in detergent free washing in washing machines and in cleaning oil-contaminated machinery. In a similar vein, U.S. Pat. No. 5,309,739 discloses a tourmaline coating applied to a surface of a washing machine with the intent of forming hydroxyl ions in the wash water to aid in cleansing. It has also been disclosed (JP 7024444) as being used in powder form in a shower apparatus to treat chlorinated water, so as to alter the chlorine structure, allowing it to retain its water-purifying activity, while reducing the possible negative effects of chlorine.

In other embodiments, glycol or its derivatives are used. Glycol derivatives include propylene glycol, hexylene glycol, ethylene glycol, diethylene glycol, and glycerin.

When preparing a composition, the weight ratio of the complex silicate to the ionizable solvent preferably ranges from about 1:5 to about 1:20. In other aspects, the weight ratio of the complex silicate to the ionizable solvent ranges from about 1:5 to about 1:10.

When preparing an ionic composition, the weight ratio of the complex silicate to the composition may range from about 0.05:1 to about 2.5:1. Alternatively, the weight ratio of the complex silicate to composition ranges from about 0.5:1 to about 1:1.

In other embodiments, a composition of the invention suitable for use as a cosmetic can be contained in a container suitable for distribution of the composition in the form of a liquid. Such a container may include an outlet for distributing the contents of the container (i.e., a cosmetic product) in the form of a liquid, and a conduit through which the liquid flows from the container to an outlet, such as a nozzle. In some embodiments, a material comprising a complex silicate is provided at least at a part of the conduit such that the material contacts an ionizable solvent. It is understood that the ionizable solvent may be admixed with other components of the composition prior to, contemporaneous with, or subsequent to contact with the material. For example, the material may be compartmentalized in the container is such a way that it first contacts only the ionizable solvent and not additional components of the composition.

Alternatively, the material and ionizable solvent may be admixed concurrently with additional components of the composition before the composition is applied to a target surface, such as the hair or skin. Those skilled in the art of cosmetic product manufacture and distribution can easily determine the spatial and temporal association of the components of the composition necessary for the desired effect on the target surface. All that is required is that the ionizable solvent be modified (e.g., ionized) through contact with the material and the beneficial effects of the contact transferred to the target surface without transferring the complex silicate associated with the material to the target surface. For example, the ionizable solvent may pass by the material as it flows toward the outlet of a container.

Accordingly, the material comprising a complex silicate may be held in a material holder which is provided at least at a part of the conduit connecting the inside of the container with the outlet. The material holder may be a cartridge provided at any point of the conduit (e.g., proximal to the inlet, equidistant from the inlet and outlet, or proximal to the outlet) and having a hollow portion in which the complex silicate-containing material is to be held. The cartridge may be detachably coupled to the conduit to facilitate the exchange of the complex silicate. As an ionizable solvent passes through the material associated with the material holder and the conduit, the solvent is ionized. Once ionized, the solvent may then be admixed with other components of the composition prior to, or contemporaneous with exiting the outlet. The complex silicate to be accommodated in the material holder may be distributed into small particles having sizes ranging from 0.1 mm to 5 mm, and more preferably, from 0.5 mm to 1 mm so that the complex silicate comes into contact with the liquid effectively.

Alternatively, the material, solvent and additional components may be present together in the container. The liquid contents of the container, in the form of an ionized composition, may then be distributed to a target surface through the conduit and outlet. The contacting of a material including a complex silicate with an ionizable solvent means that the two different elements are in proximity to one another such that the material modifies the function of the solvent.

It is further understood that a container described herein may be internally configured to accommodate the admixing, ionization and distribution of a cosmetic product prior to application to a target surface. As used herein, the term “configured” is defined as the geometry of a container is arranged so as to function in accordance with the role of the container in dispensing an ionized composition. For example, the container may be “configured” to include a single chamber where the material and composition comprising an ionizable solvent reside in specified amounts until the ionized composition is dispensed. It is understood that the container may be modified by altering, for example, the single chamber to a multi-chamber format. It is also understood that the ionized composition can include additional compounds, materials, or elements suitable for use in a cosmetic product.

Accordingly, in other embodiments the composition may further include an amphoteric surfactant, a mild anionic, a anionic sulfate, a humectant, an emollient, a viscosity modifier, a biologically active material, a foam booster, a conditioning agent, a silicone surfactant, a cationic polyquaternium surfactant, an anti-dandruff agent, a fragrance compound, or any combination thereof. Exemplary amphoteric surfactants include alkyl betaines, amido alkyl betaines, sulfobetaines, and N-alkyl-amino propionates. Exemplary anionics include fatty acid carboxylates, taurates, sulfosuccinates, isethionates, taurates, sarcosinates, and monoalkyl phosphates. Exemplary viscosity modifiers include gum, cellulose derivatives, PEG esters or carbopol resins. Exemplary biologically active materials include hydroxy acids, antiinflammatories, and an anti-irritants. Exemplary anionic sulfates include alkyl sulfates, alkyl ether sulfates, monosulfosuccinates, disulfosuccinates, alpha-olefin sulfates, and monoglyceride sulfates. Exemplary foam boosters include alkanolamides and N-alkylpyrrolidones. Exemplary conditioning agents include amine oxides. Exemplary silicone surfactants include amodimethicone and dimethicone copolyol. Exemplary anti-dandruff agents include zinc pyrithione and pyroctone olamlne.

In some embodiments, methods provided herein may further include heating the composition for a period of time ranging from about 10 seconds to about 10 minutes, or for a period of time ranging from about 30 seconds to about 1 minute, or at a temperature ranging from about 25° C. to about 75° C., or from about 30° C. to about 60° C., or from about 37° C. to about 50° C. In other embodiments, the composition may be heated under a pressure ranging from about 1 atm to about 5 atm, or from about 1 atm to about 2 atm.

Compositions of the invention are particularly useful in hair care products. Shampooing the hair strips the hair and scalp of its natural minerals, often leaving the hair looking dry and brittle, and making it very difficult to comb or brush. The use of conditioner after shampooing the hair is intended to repair some of the damage caused by the shampooing. However, the conditioner is sometimes difficult to rinse out of the hair, thereby leaving the hair with a greasy texture that adversely affects the appearance of the hair by reducing the shiny appearance of the hair.

Shampooing the hair is necessary to clean out the sebum secreted by the scalp and the dirt that accumulates in the hair. Also, conditioning is preferred in order to make the hair easier to comb or brush and facilitate easier styling of the hair. Styling of the hair often requires the use of both heat from a hair dryer or curling iron, and styling products such as hairspray, gel or mousse. This combination, however, causes damage to the hair adversely affecting its appearance. Hair care products (e.g., conditioner and shampoo) that include ionized compositions described herein are not only less damaging to the hair and scalp, but also impart desirable qualities such as sheen, luster and manageability to the hair upon application.

Accordingly, the invention further encompasses methods for cleansing, conditioning or modifying the appearance of hair, is provided. The method includes contacting the hair with a cosmetic product provided herein under conditions suitable for cleansing, conditioning or modifying the appearance of the hair. In yet another embodiment, a method of applying a hair care product that includes an ionic composition to the hair, is provided. The method includes preparing an ionic composition provided herein, adding the composition to a hair care product, thoroughly mixing the composition into the hair care product, and applying the hair care product to the hair.

Cosmetic products that includes a composition set forth herein includes a hair care product, a skin-care cream, a lotion, a body powder, a body spray, perfume, lipstick, fingernail polish, eye and facial makeup, hair color product, deodorant, bath oil and bubble bath. In some aspects, the hair care product is a shampoo, conditioner, hairspray, permanent wave product or mousse.

An exemplary cosmetic product according to the invention may be prepared by admixing cocamidopropyl hydroxysultaine (about 10-20% by weight), coco/oleamidopropyl betaine (about 10-20% by weight), TEA-Cocoyl glutamate (about 1-20% by weight), sodium cocoyl sarcosinate (about 15-25% by weight, sodium laureth sulfate (about 5-15% by weight), polyquaternium-6 (about 0.10-1.0%), methyl paraben (about 0.10-1.0% by weight), propyl paraben (about 0.10-1.0 by weight), water (QS) and a complex silicate such as tourmaline (about 0.1-5.0% by weight).

Performance test methods known to those skilled in the cosmetic industry may be used to determine the quantity and type of complex silicate needed to enhance the performance of a particular cosmetic product. For example, with regard to hair care products, a composition comprising a complex silicate and an ionizable solvent may be prepared and the application solution applied directly to the hair and scalp of a test subject. Alternatively, the composition may be admixed with a commercially available hair care product (e.g., shampoo, conditioner, mousse, hairspray, etc) and the resulting application solution applied to the hair and scalp of a test subject.

The products with the composition added may be tested alongside the same products without the composition. Each product, both with and without the composition, may then be rated separately by a plurality of skilled artisans, such as a series of professional hairstylists. Each stylist may test each product by applying the application solution resulting from the composition containing the complex silicate and ionizable solution on one half of a test subject's head, and applying the product without the composition on the other half of the test subject's head. Accordingly, the skilled artisan may rate each product according to a plurality of criteria that may be used to determine enhanced performance of the product by examination of treated hair. The criteria may include enhanced gloss, enhanced conditioning and moisturizing, reduced static flyaways, and enhanced the ease of combing and brushing. Additional criteria may include foam quality, foam quantity, fragrance during drying, ease of wet combability, ease of dry combability, residual fragrance on hair, hair sheen and luster, and feel and touch of dry hair. Finally, the evaluation may include such characteristics as the general appearance, fragrance, pourability, and ease of application of the product.

Table 1 provides data showing the advantageous effects of an exemplary cosmetic product prepared by a method of the invention. The exemplary hair care product was charged with negative and/or positive ions by contacting the product with a complex silicate as described above. In comparison to an uncharged product, the charged product produced shinier hair, promoted hair color retention, promoted closing of the cuticle layer, provided increased hair protection, pushed excess moisture, promoted hair drying, reduced static flyaway of the hair, and neutralized odors in the hair.

TABLE 1

| | Product Charged With |
|---|---|
| Characteristic/Quality | Negative and/or |
| of Hair | Positive Ions |
| Shinier hair | YES |
| Hair protection | YES |
| Promotes closing of | YES |
| cuticle layer | |
| Promotes Color retention | YES |
| Pushes excess moisture | YES |
| Promotes hair drying | YES |
| Reduces static flyaway | YES |
| hair | |
| Neutralizes odors in the | YES |
| hair | |

The examples set forth above are provided to give those of ordinary skill in the art a complete disclosure and description of how to make and use the embodiments of the devices, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains. All references cited in this disclosure are incorporated by reference to the same extent as if each reference had been incorporated by reference in its entirety individually.

A number of embodiments of the invention have been described. Nevertheless, it will be understood that various modifications may be made without departing from the spirit and scope of the invention. Accordingly, other embodiments are within the scope of the following claims.

What is claimed is:

1. A method of preparing an ionic composition suitable for use as a cosmetic product, the method comprising:
a) contacting a composition comprising an ionizable solvent with a material comprising a piezoelectric and/or pyroelectric complex silicate, wherein the ionizable solvent is at least partially ionized; and
b) separating the composition comprising the ionized solvent from the material comprising the complex silicate, wherein the composition is at least partially ionized.

2. The method of claim 1, further including heating the composition comprising the complex silicate, under pressure, prior to separation.

3. The method of claim 1, wherein the complex silicate is selected from the tourmaline mineral group (TMG).

4. The method of claim 3, wherein the complex silicate comprises the general chemical formula:
Na(Al,Fe,Li,Mg,Mn)M₃Al(Si₆O₁₈)(BO₃)₃(OH,F)₄.

5. The method of claim 4, wherein the complex silicate comprises a chemical formula selected from the group consisting of:
Na(Li_{1.5},Al_{1.5})Al₆Si₆O₁₈(BO₃)₃(OH)₄;
NaFe²⁺₃Al₆Si₆O₁₈(BO₃)₃(OH)₄;
NaMg₃Al₆Si₆O₁₈(BO₃)₃(OH)₄;
NaMg₃Cr₆Si₆O₁₈(BO₃)₃(OH)₄;
NaAl₃Al₆Si₆O₁₈(BO₃)₃O₃OH;
NaFe³⁺₃Al₆Si₆O₁₈(BO₃)₃O₃F
NaFe³⁺₃(Fe³⁺₄Mg₂Si₆O₁₈(BO₃)₃(OH)₃O;
NaMg₃V₆Si₆O₁₈(BO₃)₃(OH)₄;
Ca(Li₂Al)Al₆Si₆O₁₈(BO₃)₃(OH)₃F;
CaMg₃(MgAl₅Si₆O₁₈(BO₃)₃(OH)₃F;
CaFe²⁺₃(MgAl₅Si₆O₁₈(BO₃)₃(OH)₄;
(LiAl₂)Al₆Si₆O₁₈(BO₃)₃(OH)₄;
(Fe²⁺₂Al)Al₆Si₆O₁₈(BO₃)₃(OH)₄; and
(Mg₂Al)Al₆Si₆O₁₈(BO₃)₃(OH)₄.

6. The method of claim 5, wherein the complex silicate is selected from the group consisting of elbaite, schorl, dravite, chromdravite, olenite, buergerite, povondraite, vanadiumdravite, liddicoatite, uvite, hydroxyferuvite, rossmanite, foitite, and magnesiofoitite.

7. The method of claim 1, wherein the material comprising the complex silicate is a particle.

8. The method of claim 7, wherein the particle size ranges from about 0.1 mm to about 100 mm in diameter.

9. The method of claim 7, wherein the particle size ranges from about 0.1 mm to about 10 mm in diameter.

10. The method of claim 7, wherein the particle size ranges from about 0.1 mm to about 1 mm in diameter.

11. The method of claim 1, wherein the complex silicate is present in the material in an amount ranging from about 0.50% to about 99.5% by weight.

12. The method of claim 1, wherein the complex silicate is present in the material in an amount ranging from about 0.50% to about 50% by weight.

13. The method of claim 1, wherein the complex silicate is present in the material in an amount ranging from about 0.50% to about 25.5% by weight.

14. The method of claim 1, wherein the cosmetic product is selected from the group consisting of a hair care product, a skin-care cream, a lotion, a body powder, a body spray, perfume, lipstick, fingernail polish, eye and facial makeup, hair color product, deodorant, bath oil and bubble bath.

15. The method of claim 14, wherein the hair care product is selected from the group consisting of shampoo, conditioner, hairspray, permanent wave product and mousse.

16. The method of claim 1, wherein the ionizable solvent is selected from the group consisting of deionized water and glycol or its derivatives.

17. The method of claim 16, wherein the ionizable solvent is deionized water.

18. The method of claim 1, wherein the composition further comprises an amphoteric surfactant, a mild anionic, a anionic sulfate, a humectant, an emollient, a viscosity modifier, a biologically active material, a foam booster, a conditioning agent, a silicone surfactant, a cationic polyquaternium surfactant, an anti-dandruff agent, a fragrance compound, or any combination thereof.

19. The method of claim 18, wherein the amphoteric surfactant is selected from the group consisting of alkyl betaines, amido alkyl betaines, sulfobetaines, and N-alkyl-amino propionates.

20. The method of claim 18, wherein the mild anionic is selected from the group consisting of fatty acid carboxylates, taurates, sulfosuccinates, isethionates, taurates, and sarcosinates, and monoalkyl phosphates.

21. The method of claim 18, wherein the viscosity modifier is selected from the group consisting of gums, cellulose derivatives, PEG esters and carbopol resins.

22. The method of claim 18, wherein the biologically active material is selected from the group consisting of hydroxy acids, antiinflammatories, and anti-irritants.

23. The method of claim 18, wherein the anionic sulfate is selected from the group consisting of alkyl sulfates, alkyl ether sulfates, monosulfosuccinates, disulfosuccinates, alpha-olefin sulfates, and monoglyceride sulfates.

24. The method of claim 18, wherein the foam booster is selected from the group consisting of alkanolamides and N-alkylpyrrolidones.

25. The method of claim 18, wherein the conditioning agent is an amine oxide.

26. The method of claim 18, wherein the silicone surfactant is selected from the group consisting of amodimethicone and dimethicone copolyol.

27. The method of claim 18, wherein the anti-dandruff agent is selected from the group consisting of zinc pyrithione and pyroctone olamlne.

28. The method of claim 2, wherein the composition is heated for a period of time ranging from about 10 seconds to about 10 minutes.

29. The method of claim 28, wherein the composition is heated for a period of time ranging from about 30 seconds to about 1 minute.

30. The method of claim 2, wherein the composition is heated at a temperature ranging from about 25° C. to about 75° C.

31. The method of claim 30, wherein the composition is heated at a temperature ranging from about 30° C. to about 60° C.

32. The method of claim 31, wherein the composition is heated at a temperature ranging from about 37° C. to about 50° C.

33. The method of claim 2, wherein the composition is heated under a pressure ranging from about 1 atm to about 5 atm.

34. The method of claim 33, wherein the composition is heated under a pressure ranging from about 1 atm to about 2 atm.

35. A cosmetic product comprising an ionic composition prepared by the method of claim 1.

36. A method for cleansing, conditioning or modifying the appearance of hair, the method comprising contacting the hair with the cosmetic product of claim 35 under conditions suitable for cleansing, conditioning or modifying the appearance of the hair.

37. A method of applying a hair care product comprising an ionic composition to the hair, the method comprising:
a) preparing the ionic composition of claim 1;
b) adding the composition to a hair care product;
c) thoroughly mixing the composition into the hair care product; and
d) applying the hair care product to the hair.

38. A container for containing and dispensing a cosmetic product comprising:
a) a container comprised of at least one chamber comprising a composition comprising an ionizable solvent associated with a material comprising a piezoelectric and/or pyroelectric complex silicate, wherein the ionizable solvent is at least partially ionized;
b) a mechanism for separating the composition comprising the ionized solvent from the material comprising the complex silicate, wherein the composition is at least partially ionized; and
c) an outlet for dispensing the ionized composition.
